(19) United States
(12) Patent Application Publication
(10) Pub. No.: US 2024/0238583 A1
(43) Pub. Date: Jul. 18, 2024

(54) ELECTRICAL STIMULATION DEVICES AND RELATED CONTROLLERS AND METHODS

(71) Applicant: Axogen Corporation, Alachua, FL (US)

(72) Inventors: Brian ROMOT, Alachua, FL (US); Kasra TAJDARAN, Alachua, FL (US); Justin DEUERLING, Alachua, FL (US); Curt DEISTER, Alachua, FL (US)

(73) Assignee: Axogen Corporation, Alachua, FL (US)

(21) Appl. No.: 18/413,173

(22) Filed: Jan. 16, 2024

Related U.S. Application Data
(60) Provisional application No. 63/480,318, filed on Jan. 18, 2023.

(51) Int. Cl. A61N 1/04

(52) U.S. Cl. CPC A61N 1/0472

(57) ABSTRACT

An electrical stimulation device may include a handle having a proximal end, a distal end, one or more actuators, and a display. The electrical stimulation device may also include a probe attached to and extending from the distal end of the handle, the probe including a stimulation portion and a return portion, wherein a spacing between the stimulation portion and the return portion is adjustable.

### CROSS-REFERENCE TO RELATED APPLICATIONS

This patent application claims the benefit under 35 U.S.C. § 120 to U.S. Provisional Patent Application No. 63/480,318, filed on Jan. 18, 2023, the entirety of which is incorporated herein by reference.

### FIELD OF THE INVENTION

The present disclosure relates generally to the field of electrical stimulation devices for use in nerve location, diagnostic and/or outcome measurement evaluations, stimulation and regeneration, and/or pain management, and a related controller and method. More particularly, the present disclosure relates to an electrical nerve stimulation device having a handle and a probe for delivering electrical stimulation.

### BACKGROUND

Electrical stimulation devices are used to provide electrical stimulation to tissue for a number of reasons, such as locating or identifying a particular tissue, such as a nerve, assessing and/or diagnosing injuries to tissue, treating injured tissue, and/or providing therapy to the tissue. Existing electrical stimulation devices are designed for a specific use, among those listed, and, therefore, a user may have to use several electrical stimulation devices during an operation, which is inconvenient, costly, and increases the time required to complete the operation. In some aspects, existing electrical stimulation devices may lack versatility. Thus, the user needs to switch between electrical stimulation devices depending on the task at hand.

Existing electrical stimulation devices may also require use of a grounding needle, when used for electrical stimulation of a nerve and/or locating a nerve. The grounding needle can be difficult to use and introduces a sharp into an area surrounding the nerve, which is not necessary.

In view of the foregoing problems, there is a need for a relatively more versatile and easy to use electrical stimulation device, having multiple functions that allow a user to attach a probe for a particular task or purpose, and that includes a safer ground portion, without a sharp grounding needle.

The present disclosure provides solutions to one or more of the problems discussed above.

### SUMMARY

In accordance with some aspects of the present disclosure, an electrical stimulation device may include a handle including a proximal end, a distal end, one or more actuators, and a display. The electrical stimulation device may also include a probe attached to and extending from the distal end of the handle, the probe including a stimulation portion and a return portion, wherein a spacing between the stimulation portion and the return portion is adjustable.

Other objects, features and advantages of the present disclosure will become apparent from the following detailed description. It should be understood, however, that the detailed description and the examples, while indicating exemplary embodiments of the present disclosure, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of exemplary embodiments presented herein.

FIG. 1 is a schematic view of an electrical stimulation device according to one or more embodiments.

FIG. 2 is a schematic detail view of a probe tip of the electrical stimulation device according to one or more embodiments.

FIG. 3 is a schematic view of a forceps probe of the electrical stimulation device according to one or more embodiments.

FIG. 4 is a schematic detail view of a concentric probe tip of the electrical stimulation device according to one or more embodiments.

FIG. 5 is a cut-away schematic view of a portion of a handle of the electrical stimulation device according to one or more embodiments.

FIG. 6 is a schematic view of discrete actuators of the electrical stimulation device according to one or more embodiments.

FIG. 7 is a schematic diagram of a controller for the electrical stimulation device according to one or more embodiments.

FIG. 8 is a flowchart of a method of controlling the electrical stimulation device according to one or more embodiments.

### DETAILED DESCRIPTION

In this disclosure, the singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a composition, method, or process that comprises a list of elements or steps does not necessarily include only those elements or steps, but may include other elements or steps not expressly listed or inherent to such a composition, method, or process. The relative terms, such as “approximately” and “about,” are generally used to indicate a possible variation of +10% of a stated or understood value unless indicated otherwise in the specification. In addition, the term “between” used in describing ranges of values is intended to include the minimum and maximum values described herein. The use of the term “or” in the claims and specification is used to mean “and/or” unless explicitly indicated to refer to alternatives only or the alternatives are mutually exclusive, although the disclosure supports a definition that refers to only alternatives and “and/or.” As used herein “another” may mean at least a second or more. The term “proximal” is used to define a region of the device located closer to a user, and the term “distal” is used to define a region located closer to a subject to which electrical stimulation is applied.

The present invention is directed to an electrical stimulation device, a controller for an electrical stimulation device, and a related method for an electrical stimulation device. In some aspects, the electrical stimulation device may comprise a handle that includes a proximal end, connected to a first wire and a second wire, a distal end, one or more actuators (e.g., control buttons), and a display. The first wire and the second wire may each be capable to act as one or both of: an electrical supply wire and a return wire. The electrical stimulation device may also include an interchangeable probe attached to and extending from the distal end of the handle. The probe may include a stimulation portion, connected to the first supply wire, and a return portion, connected to the second wire. In related aspects, a controller is provided for an electrical stimulation device having a handle including a proximal end connected to a first wire and a second wire, a distal end, one or more actuators and a display, and a probe attached to and extending from the distal end of the handle. The probe may be movable and/or interchangeable (e.g., configured for replacement with a probe to modify the functionality of the device and/or for a subsequent use of the device, or to facilitate sterilization of the device). The probe may include a stimulation portion, connected to the first wire, and a return portion, connected to the second wire. The controller may comprise at least one memory that stores instructions, and at least one processor configured to execute the instructions to perform a method comprising controlling supply of electricity to the stimulation portion of the probe. These and other aspects are described below in more detail.

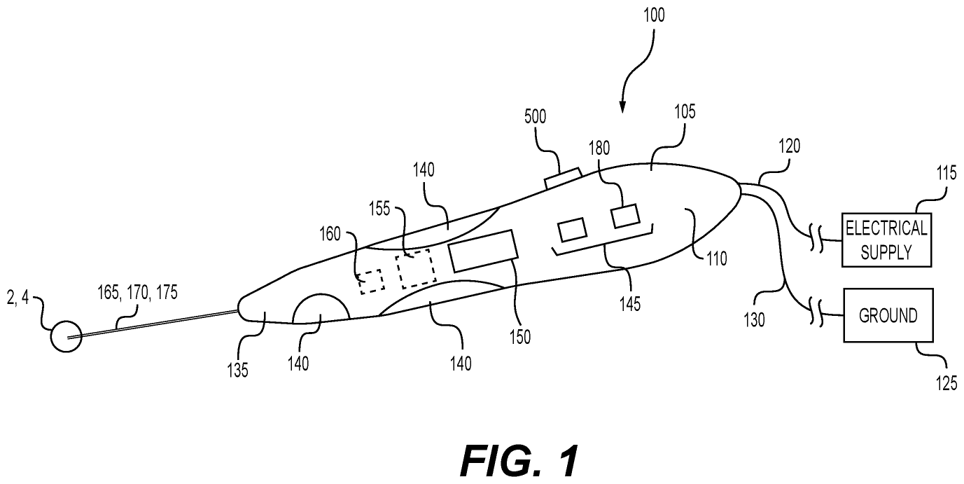

FIG. 1 is a schematic view of an electrical stimulation device 100 according to one or more embodiments. The electrical stimulation device 100 may include a handle 105 having a proximal end 110, which is configured to be connected to an electrical supply 115 via at least one of a first wire 120 and a second wire 130. First wire 120 may be configured to act as a supply wire and second wire 130 may be configured to operate as a return wire. As understood, during at least some modes of operation, wires 120 and 130 may repeatedly alternate between acting as supply and return wires.

Additionally, while electrical supply 115, ground 125, and wires 120 and 130 are shown as being external to the handle 105, in at least some embodiments, one or more of these components may be enclosed within handle 105 (e.g., a battery that forms electrical supply 115 may be secured within handle 105).

In some embodiments, the proximal end 110 may be connected to an electrical ground 125 via the second wire 130. The electrical stimulation device 100 also includes a distal end 135 at which a probe (e.g., any one of the probes described herein) may be attached. In some embodiments, the handle 105 may be curved for an ergonomic fit with a hand of a user, and may have one or more grips 140 at one or more locations to improve handling control for a user. Grips 140 may be formed a conformable material, such as soft rubber or plastic, and may form protrusions that are raised slightly from the housing of device 100. Further, the handle 105 may be weighted at a proximal or central region, providing a more ergonomic device to reduce user fatigue.

The handle 105 may also have one or more actuators 145, e.g., control buttons, including an ON/OFF button 180, as shown in FIG. 1, a slider 500 (also shown in FIG. 5), a release (e.g., a button, as shown in FIG. 5) for detachment of probe 165 (in embodiments in which probe 165 is interchangeable), a variable actuator, and/or a discrete actuator, as described below. The ON/OFF button 180 may function as a switch that, when switched on or depressed, for example, causes the generation of one or more stimulation pulses (that is, closes a circuit with the leads) via probe 165, and when released, discontinues the generation of stimulation pulses (that is, opens the circuit with the leads). Functions of one or more of these actuators 145 may be combined into a single button, for example, a button that controls both ON/OFF of the electrical stimulation device 100 may be the same button that controls a supply of energy to the electrical stimulation device 100. Other actuators may include, for example, one or more knobs, levers, switches, rotatable wheels, touch screens, etc. The actuators 145 may be placed in easy-to-reach locations on the handle 105 for a user, based on a need of the user to access the actuators 145 during or outside of use.

The handle 105 may further include display 150, which may be a liquid crystal display (LCD), and which is configured to display information to a user of the electrical stimulation device 100. For example, the display 150 may display text indicating one or more variable settings, such as “Stim On/Off” (indicating whether the device 100 is on or off), electrode spacing, pulse duration, frequency, voltage, and/or amplitude of current. The displayed text may indicate whether a nerve has been identified. The text may be monochromatic (e.g., all black, all green, all blue, etc.) or in multiple colors. The display 150 may also or alternatively be a light emitting diode (LED) display, an organic LED display, or other suitable display for providing information on the display 150 for the user. The handle 105 may also contain a controller 155 and a motor 160 (both shown in dashed lines). The controller 155 may be used in controlling supply of electricity to the probe 165 and, along with the motor 160, may be used in controlling a spacing between leads of the probe 165, as discussed in more detail below.

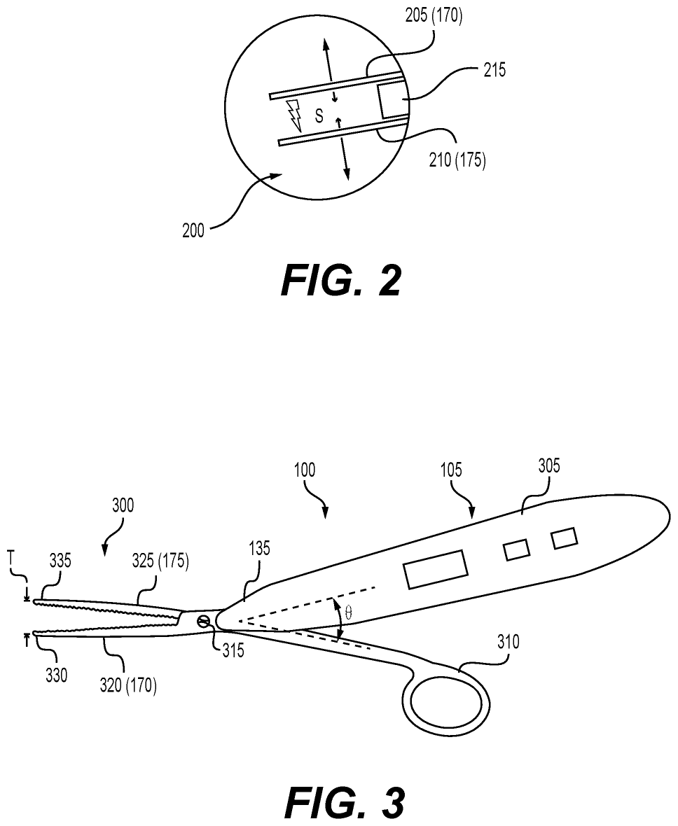

In some configurations, the device 100 may enable electronic control of one or more aspects of probe 165. For example, device 100 may, via controller 155, control and adjust a spacing S (FIG. 2) between different portions of probe 165. Device 100 may, additionally or alternatively, control whether probe 165 acts as a monopolar probe or a bipolar probe. The spacing S of probe 165 may be modified by control of motor 160 to move one portion of probe 165 (e.g., one probe tip) in a direction away from an opposing portion of probe 165 (e.g., a second probe tip), as described below.

In some aspects, probe 165 may be removably coupled to electrical stimulation device 100, allowing probe 165 to be interchangeable. The probe 165 may be removably attached to and extend from the distal end 135 of the handle 100. An interchangeable probe 165 may allow a user to change between different probe heads on the electrical stimulation device 100. The probe 165 may be detached from the handle 105 by actuating an actuator, e.g., a release button (such as a quick release button), such as one of the actuators 145 provided on the handle 105. Once a probe 165 is released from the distal end of the electrical stimulation device 100, another probe may be attached to the distal end 135 of the handle 105.

In some aspects, the electrical stimulation device 100 may provide monophasic stimulation and biphasic stimulation, in a single device, to a nerve or other tissue to be located or otherwise stimulated using the electrical stimulation device 100. Monophasic waveforms delivered with electrical stimulation device 100 may include waveforms having pulses that are positive or negative, but that do not switch between positive and negative. For example, pulses of a monophasic waveform may each be a +5 volts (V) pulse. Biphasic stimulation with electrical stimulation device 100 may include generating waveforms that alternate between positive and negative using probe 165 (see FIG. 2).

Additionally or alternatively, the electrical stimulation device may be configured to provide monopolar stimulation and/or bipolar stimulation via probe 165. For example, a user may interact with an actuator on handle 105 to select between monopolar stimulation and bipolar stimulation. Bipolar stimulation may enable the use of separate electrodes at the distal end 135 of the handle 105 to supply bipolar energy (e.g., supply portion 205 and return portion 210, as shown in FIG. 2 and described below). Monopolar stimulation may involve the use of a return electrode placed on the subject to complete a circuit, while the electrode(s) at distal end 135 operate to supply monopolar energy. When multiple electrodes are included at distal end 135, a monopolar mode may involve disabling one or more of these electrodes or using multiple electrodes in a monopolar mode where energy is not returned via electrodes at distal end 135, but instead by the return electrode on the subject, for example.

Whether configured for monopolar stimulation or bipolar stimulation (or configured to enable a user to select between monopolar and bipolar modes), electrical stimulation device 100 may be configured to generate biphasic waveforms that avoid overstimulation of the target tissue. For example, pulses of a biphasic waveform may include a first pulse in a first (e.g., anodal) direction and a second pulse in a second (e.g., cathodal) direction, via respective power sources. The first pulse may be, for example, a +5 volts (V) pulse and the second pulse may be a −5 V pulse. These phases may be symmetric or asymmetric, and may have the same (but opposite) amplitudes and durations.

In configurations where monopolar and/or bipolar probes 165 are replaceable, electrical stimulation device 100 may detect the type of probe that is attached to the electrical stimulation device 100, patch, etc.) and may automatically switch to a corresponding configuration. For example, if a monopolar probe is attached, the electrical stimulation device 100 may detect the presence of the monopolar probe and may configure itself for monopolar use. Automatic detection of the presence of the monopolar probe or the bipolar probe may be achieved by detecting a change in a property of the circuit that is formed when the monopolar probe or the bipolar probe is attached to the electrical stimulation device 100. For example, a resistance of an attached probe may be detected, and the monopolar probe and the bipolar probe may have different resistances that allow electrical stimulation device 100 to determine which probe has been attached to the electrical stimulation device 100 by detecting the resistance of the attached probe. In other aspects, a current or a voltage of an attached probe may be detected, and the monopolar probe and the bipolar probe may have different currents or voltages that allow electrical stimulation device 100 to determine which probe has been attached to the electrical stimulation device 100 by detecting the current or voltage of the attached probe. A change in one or more of resistance, current, voltage, or other property in a circuit that is formed when the monopolar probe or the bipolar probe is attached may indicate to the electrical stimulation device 100 which probe has been attached. Based on the property of the circuit detected, the electrical stimulation device 100 may automatically configure itself for either bipolar or monopolar use.

In some embodiments, automatic detection of the presence of the monopolar probe or the bipolar probe may be achieved by a unique wiring connection formed when the monopolar probe or the bipolar probe is attached to the device electrical stimulation 100. For example, a first circuit may be completed when a monopolar probe is attached, and a second, different, circuit may be completed when a bipolar probe is attached. In some embodiments, the positioning of the probe tips of the electrical stimulation device 100 may be detected and used to determine whether the electrical stimulation device 100 should be in a monopolar or a bipolar mode. In some aspects, if a single probe may be used in either bipolar or monopolar modes, the electrical stimulation device 100 may be able to identify when the bipolar probe tips have been aligned with one another, which may trigger a switch into a monopolar mode, instead of a bipolar mode when the probe tips are not aligned with/are spaced apart from one another. In some aspects, the electrical stimulation device 100 may detect whether the grounding electrode is electrically connected or is in use to determine whether the electrical stimulation device 100 should operate in a bipolar or monopolar mode.

In other embodiments, the user may manually switch between the monopolar function and the bipolar function, depending on a desired mode of operation and/or depending on which probe 165 is present. For example, a user may make a mechanical selection, such as moving an actuator that opens or closes an appropriate circuit (e.g., either closes a bipolar circuit or a monopolar circuit) within the electrical stimulation device 100 for use with a monopolar probe or a bipolar probe.

One or more components of device 100 may be capable of desirable performance in multiple use cases. For example, device 100 may be capable of nerve location when provided with a first probe 165 (e.g., a probe 165 with portions 170 and/or 175 for handheld use), nerve regeneration when provided with a second probe 165, and/or nerve pain blocking when provided with a third probe 165. Thus, a probe 165 may be configured to perform one or more particular functions, such as nerve location, regeneration treatment, or pain management. In some aspects, device 100 may be capable of bipolar use when a first probe 165 is attached, or may be capable of monopolar use when a second probe 165 is attached. In other aspects, the same probe 165 may be switched from monopolar to bipolar use.

When probe 165 is interchangeable, the probe 165 for a desired use case may be attached to the handle 105, and thus, electrical stimulation device 100 may be capable of multiple functions from a single device, to allow a user to select a desired probe for a task at hand without needing to switch devices and use multiple devices in one operation. Interchangeability of probe 165 may also facilitate sterilization of the probes or may allow for single-use probes in combination with a reusable body 105.

The probe 165 includes a stimulation portion 170 (FIG. 2), also referred to as a source lead, which operably connects to the first wire 120 within the handle 105 at a proximal region. The probe 165 also includes a return portion 175 (FIG. 2), or a sink lead, which connects to the second wire 130 within the handle 105. In one embodiment, the electrical stimulation device 100 includes one probe as the stimulation portion 170, and another probe for the return portion 175, in other words, a stimulation probe 170 and a return probe 175, respectively, thereby forming a double (e.g., two-pronged) probe 165. In another embodiment, the stimulation portion 170 and the return portion 175 are included as separate poles on a single (e.g., one-pronged) probe. In either the double probe or the single probe embodiment, it is possible to manipulate or change a distance or a spacing between the stimulation portion 170 and the return portion 175, as discussed in more detail below, for more precise control of electrical stimulation. In some aspects, the distance or spacing between the stimulation portion 170 and the return portion 175 may be changed in a continuous manner or in a discrete manner. For example, if the latter, there may be discrete distance or spacing settings that a user may choose between.

FIG. 2 is a schematic detail view of one example of a probe tip 200 of the electrical stimulation device 100 shown in FIG. 1. In particular, FIG. 2 shows a stimulation portion 205, as the stimulation portion 170, and a return portion 210, as the return portion 175, extending parallel to one another. The probe tip 200 is a bipolar probe. Although a parallel configuration is shown, stimulation portion 205 and return portion 210 may be angled towards or away from one another. A distance or a spacing S between the stimulation portion 205 and the return portion 210 may be adjustable, as indicated by the outwardly-and-inwardly pointing arrows in FIG. 2. The spacing S between the stimulation portion 205 and the return portion 210 may be changed by an actuator, e.g., a slider (as described below), a wheel, a knob, a lever, a switch, a trigger, or a touch-screen. The spacing S between the electrodes, that is, between the stimulation portion and the return portion, may be changed for nerve identification, and, more particularly, for identification of different types of nerves, different sized nerves, different locations on the body, etc., or identification of fascicles of nerves, including individual fascicles. As discussed above, the spacing S between the electrodes may be changed continuously, or there may be discrete spacing settings that a surgeon may choose between. If the latter, for example, in the context of use with nerves, a user may be able to choose between a first spacing setting configured to provide fascicle-level stimulation, a second spacing setting configured to provide nerve-level stimulation, and/or a third spacing setting configured to provide field-level stimulation. Although this example describes three spacing setting options, more or fewer discrete spacing settings may be provided. For either continuous or discrete embodiments, in some aspects, types of individual fascicles may be identified, such as fascicles having a certain size (e.g., diameter or width) or a certain shape (e.g., circularity), etc. In addition, a spacer, e.g., insulator 215, may be provided between the stimulation portion 205 and the return portion 210, at any point along a length of these elements, with the spacer acting as a stop and preventing contact between the two elements. In some embodiments, one of the stimulation portion 205 and return portion 210 may be movable, while the other is fixed in place.

By virtue of the bipolar leads, that is, the stimulation portion 170, or source lead, and the return portion 175, or sink lead, and the capability of manipulating the spacing S therebetween, more precise control of electrical stimulation by the electrical stimulation device 100 is possible. In particular, for example, if a user has identified a potential nerve, the user can narrow or reduce the spacing S between the stimulation lead and the return lead, to stimulate only the nerve and little or none of the surrounding tissue. As another example, if a nerve cannot be identified, and a wider stimulus field (that is, a relatively wider area for electrical stimulation) is desired, the user can increase the spacing S to stimulate more tissue and to locate the nerve. A user may, for example, select or modify the spacing S based on the anatomical location where the electrical stimulation device 100 is being used (e.g., increasing the spacing S at locations where relatively larger nerves are present and reducing the spacing S at locations where smaller nerves are present). Moreover, the electrical stimulation device 100 described herein may eliminate the need for a separate, sharp ground needle, thus simplifying a process of stimulating a nerve or locating a nerve, and making the process more safe.

An insulator 215 may be provided between at least a portion of the stimulation portion 205 and the return portion 210. The insulator may be formed of DEHP-free polyvinyl chloride (PVC), polyethylene (PE), polytetrafluoroethylene (PTFE), ethylene propylene rubber (EPR), and silicone rubber, for example, although insulators formed of other insulating materials or combinations of insulating materials may be used.

The probe tip 200 may be used for location and/or stimulation of tissue, such as a nerve. In particular, for example, if a user would like to locate a nerve, the user may select an amount of electricity to be supplied to the stimulation portion 205. The user may then touch the probe tip 200 to tissue and, if the electricity flows through the return portion 210, the controller 155 may confirm that tissue, such as a nerve, has been identified. Additionally or alternatively, in some embodiments, a muscle enervated by the nerve may react (the reaction being observed by the user), then the user may confirm that the tissue contacted by the probe tip 200 is the nerve, thus locating the nerve. If the nerve cannot be located or identified, the spacing between the stimulation portion 205 and the return portion 210 may be increased, or widened, to provide a wider stimulus field, and to stimulate more tissue, to locate the nerve. And, if a user has identified the nerve, they can narrow the distance to stimulate only the nerve, to ensure stimulation of the nerve without damage to any surrounding tissues. In addition, the device 100 may be configured to provide an indication that current is being returned via one of the electrodes, so that a user can confirm that effective stimulation of a nerve is occurring. The indication may be via text or graphics in the display 150, via tactile feedback (e.g., vibration), and/or via auditory feedback. By virtue of the probe tip 200 of this embodiment, it is possible to eliminate the need for a grounding needle, thus providing a more simple and safe device for tissue location and stimulation. Further, device 100 may be turned off from providing current and may be also used to manipulate tissue or perform other functions during surgery.

FIG. 3 is a schematic view of an example of an electrical stimulation device in the form of a forceps probe 300. The forceps probe 300 may be an interchangeable probe connectable to a handle, such as handle 105 of the embodiment shown in FIG. 1. The forceps probe 300 may include a first handle portion 305 and a second handle portion 310, which move relative to each other to allow the forceps probe 300 to function as a forceps. One of the first handle portion 305 and the second handle portion 310 may be formed with one or more components of handle 105 as shown in FIG. 1 and described above. In some configurations, one or more components of handle 105 may be connected to first handle portion 305 or second handle portion 310, instead of forming the handle portion itself.

The first handle portion 305 and the second handle portion 310 meet at a connection point 315, which may be a pivot point. The first handle portion 305 may connect to a stimulation portion 320, as stimulation portion 170, at a distal end thereof, and the second handle portion 310 may connect to a return portion 325, as return portion 175, at a distal end thereof. A spacing T between a distal end 330 of the stimulation portion 320 and a distal end 335 of the return portion 325 may be controllable via the first handle portion 305 and the second handle portion 310. That is, by bringing the first handle portion 305 and the second handle 310 portion closer together (or decreasing an angle θ between the first handle portion 305 and the second handle portion 310), the spacing T between the distal end 330 of the stimulation portion 320 and the distal end 335 of the return portion 325 can be decreased. And by bringing the first handle portion 305 and the second handle portion 310 further apart (or increasing the angle θ between the first handle portion 305 and the second handle portion 310), the spacing T between the distal end 330 of the stimulation portion 320 and the distal end 335 of the return portion 325 can be increased. In addition, a locking mechanism (not shown) may be provided to maintain the spacing T between the distal ends 330 and 335.

Similarly to probe tip 200, the forceps probe 300 may be used for location and/or stimulation of tissue, such as a nerve. In particular, for example, if a user would like to locate a nerve, the user may select an amount of electricity to be supplied to the stimulation portion 320 (e.g., by interacting with components of handle 105 as described above). The user may then touch the distal ends 330, 335 of the forceps probe 300 to tissue and, if the electricity flows through the return portion, the controller 155 may confirm that tissue, such as a nerve, has been identified. Additionally or alternatively, in some embodiments, a muscle enervated by the nerve may react (the reaction being observed by the user), then the user may confirm that the tissue contacted by the forceps probe 300 is the nerve, thus locating the nerve. If the nerve cannot be located or identified, the spacing between the stimulation portion 320 and the return portion 325 may be increased, or widened, to provide a wider stimulus field, and to stimulate more tissue, to locate the nerve. And, if a user has identified the nerve, they can narrow the distance to stimulate only the nerve and nothing else, to ensure stimulation of the nerve without damage to any surrounding tissues. Further, forceps probe 300 may be turned off from providing current and may be also used to manipulate tissue or perform other functions during surgery.

By virtue of the forceps probe 300 shown in FIG. 3, which can be integrated with and/or attached to the handle 105, it is possible to provide an electrical stimulation device 100 capable of functioning as a forceps, and which also provides for location and stimulation of tissue. As with the other embodiments of the electrical stimulation device 100 with an interchangeable probe described herein, the electrical stimulation device 100 with the forceps probe 300 provides a user with a probe suited for a particular use, without requiring the user to switch tools when trying to locate and identify, treat, or manage pain for nerves.

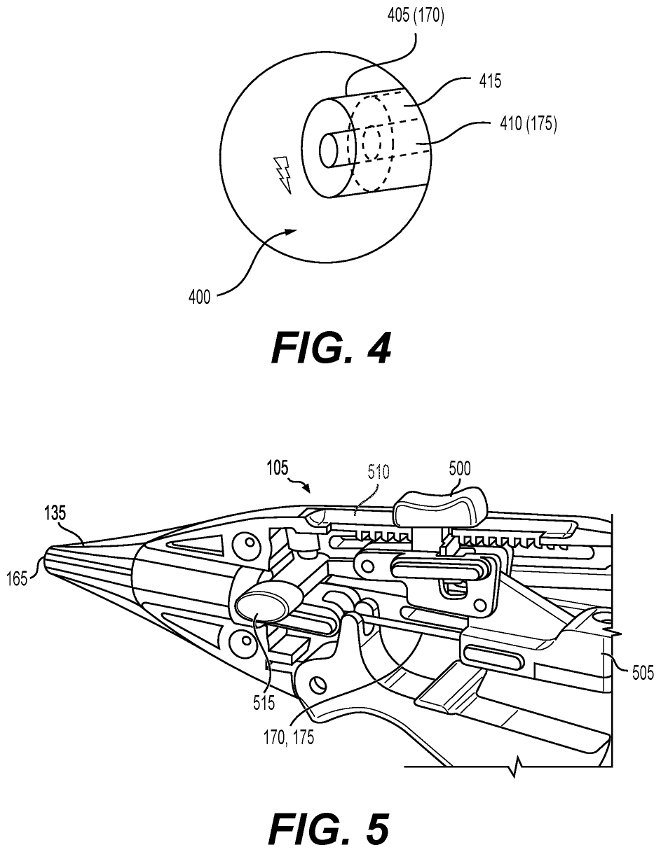

FIG. 4 is a schematic detail view of a probe tip 400 of the electrical stimulation device 100 shown in FIG. 1, with a stimulation portion 405, as stimulation portion 170, and a return portion 410, as return portion 175, being concentric. That is, the stimulation portion 405 may be formed as a hollow tubular member, and the return portion 410 may be a cylindrical rod that extends within the hollow tubular stimulation portion 410. Alternatively, the return portion 410 may be formed as a hollow tubular member, and the stimulation portion 405 may be a cylindrical rod that extends within the hollow tubular return portion 410. In either case, an insulator 415 may also be provided between at least a portion of the stimulation portion 405 and a portion of the return portion 410.

Similarly to probe tip 200, the probe tip 400 may be used for location and/or stimulation of tissue, such as a nerve. In particular, for example, if a user would like to locate a nerve, the user may select an amount of electricity to be supplied to the stimulation portion 405. The user may then touch the probe tip 400 to tissue and, if the electricity flows through the return portion 410, the controller 155 may confirm that tissue, such as a nerve, has been identified. Additionally or alternatively, in some embodiments, a muscle enervated by the nerve may react (the reaction being observed by the user), then the user may confirm that the tissue contacted by the probe tip 400 is the nerve, thus locating the nerve. If the nerve cannot be located or identified, the spacing between the stimulation portion 405 and the return portion 410 may be adjusted to provide a wider stimulus field, and to stimulate more tissue, to locate the nerve. In particular, for example, one of the stimulation portion 405 and the return portion 410 may be movable axially (that is, along a longitudinal axis), while the other one of the stimulation portion 405 and the return portion 410 may remain in its original position. And, if a user has identified the nerve, they can adjust the spacing to stimulate only the nerve and nothing else, to ensure stimulation of the nerve without damage to any surrounding tissues.

FIG. 5 is a cut-away schematic view of a portion of the handle 105 of an exemplary electrical stimulation device 100 according to one embodiment. In this embodiment, the actuator is in the form of a manually actuatable slider 500 on the handle 105, which may be used, e.g., in conjunction with the probe tip 400 of FIG. 4. The slider 500 is one example of a mechanical feature that may be used to adjust the spacing S, but as discussed in more detail below, a controller and/or a motor may be used. An inner portion 505 of the slider 500 is connected to at least one of the stimulation portion 405 (170) and the return portion 410 (175), or both, such that movement of the slider 500 causes the spacing S between the stimulation portion 170 and the return portion 175 of the probe 165 to change. This change may be in a direction approximately perpendicular to an axis defined by stimulation portion 205 and return portion 210 (FIG. 2).

With reference to the exemplary probe tip 400 shown in FIG. 4, moving the slider 500 in a distal direction, towards the distal end 135 of the handle, may cause the stimulation portion and the return portion to move further from or closer to one another in an axial direction, increasing or decreasing the spacing S. Moving the slider 500 in a proximal direction, towards the proximal end 110 of the handle 105 (shown in FIG. 1), may cause the stimulation portion and the return portion to move further from or closer to one another, increasing or decreasing the spacing S. Slider 500 may similarly control the spacing S for parallel electrodes, as indicated above. The range of the spacing S may be, for example, about 0.1 mm, as a minimum value, to about 50 mm, as a maximum value. The slider 500 may slide within a groove 510 provided in the handle 105. The slider 500 may be a continuous slider or may have pre-defined stopping points.

FIG. 5 also shows an example of a release button 515, as one of the actuators 145 provided on the handle 105. Although the term “button” is used when describing the release button 515, this element could be something other than a button, such as a trigger, a latch, a lever, a knob, a switch, a wheel, a touch screen, or a similar mechanism. The release button 515 may be connected to the probe 165 such that pressing the release button causes the probe 165 to detach from the distal end 135 of the handle 105. As a result, another interchangeable probe may be attached to the distal end 135 of the handle 105.

In some embodiments, the slider 500 may be used as a variable actuator of the electrical stimulation device 100. The slider 500 may enable a user to provide input to the controller 155 of the electrical stimulation device 100. For example, a user may change the position of slider 500 to select a value between a minimum value and a maximum value. The value may be, for example, a pulse width, a frequency, and/or an amplitude of the current supplied to the stimulation portion. A range of current supplied to the stimulation portion may be, for example, from about 0.1 mA to about 20 mA, or, more particularly, from about 0.1 mA to about 6.0 mA. A range of frequency of the electricity supplied to the stimulation portion may be, for example, from about 15 Hz to about 1 kHz. This range may be, for example, a range used for pain management. Alternatively, the range of frequency of the electricity supplied to the stimulation portion may be, for example, from about 15 Hz to about 25 Hz, or, more particularly, from about 18 kHz to about 22 kHz. This relatively narrower range may be used, for example, for nerve location and for nerve regeneration, in particular. As a specific example, sliding the slider 500 towards the distal end 135 of the handle 105 may allow a user to select an increased value as an input for the electrical stimulation device 100. And sliding the slider 500 towards the proximal end 110 (shown in FIG. 1) of the handle 105 may allow the user to select a decreased value as an input for the electrical stimulation device 100, or vice versa. The values that may be input using the slider 500 may be any within a range, allowing the user precise, continuous control over one or more parameters of the signal supplied via probe 165. For example, if a range of values, such as 0 to 200, a user may slide the slider 500 so that an input is 105. Optionally, however slider 500 may slide in preset increments providing discrete control over one or more values.

In some embodiments, the slider 500 may be associated with different discrete settings that impact multiple aspects of the device's operation, or, in other words, the slider 500 may be used to select different modes for the device 100 with controller 155. For example, the slider 500 may be moved to a first position, position 1, corresponding to a gross nerve location mode, in which an electrode distance (or spacing) can be changed to a set distance by the controller 155 via the motor 160 with controller 155 as described above. This position may also set one or more electrical parameters, such as pulse width, current amplitude, frequency, etc.

The slider 500 may also be moved to a second position, position 2, corresponding to a fine nerve location mode, in which a user may set a relatively smaller electrode distance (or spacing) (that is, the electrodes may move closer together by use of the motor 160), and set one or more other electrical parameters, such as pulse width, current amplitude, frequency, etc., including changing one or more of these parameters by a relatively small amount. The changed electrical parameters may be associated with the supply of a larger amount of electrical energy as compared to the set values in the gross nerve location mode, by increasing pulse width duration, current amplitude, and/or frequency. As a further example, the slider 500 may be moved to a third position, position 3, corresponding to a nerve regeneration mode, in which a user may place electrodes for nerve regeneration and the controller 155 may check for correct electrodes (e.g., confirm the presence of percutaneous or transcutaneous electrodes); move electrodes, and/or set or change one or more electrical parameters, such as pulse width, current amplitude, frequency, etc., including changing one or more of these parameters by a relatively large amount. Still further, the slider 500 may be moved to a fourth position, position 4, corresponding to a pain blocking mode, in which a user may place electrodes to effect pain blocking of a nerve (e.g., a percutaneous or transcutaneous electrodes), the presence of suitable electrodes being confirmed with controller 155. In any of the above-described modes, controller 155 may be configured to generate a notification (e.g., an error message, an auditory tone, etc.) when the incorrect electrodes are installed and/or may disable the ability of the device 100 to supply an electrical signal to the electrodes.

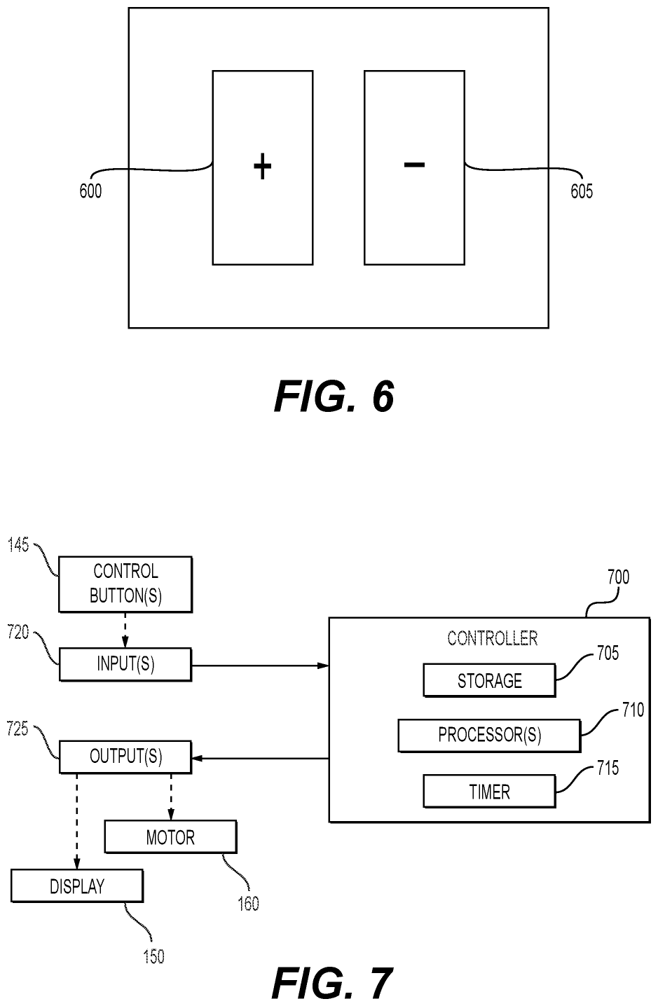

In some embodiments, the one or more of the actuators may include control buttons 600, 605, shown in FIG. 6, provided in the electrical stimulation device 100 to receive input from a user of the electrical stimulation device 100, with the input being a value between a minimum value and a maximum value. The value may be, for example, a pulse width or an energy amount, such as current, a frequency, and/or an amplitude, of the electricity supplied to the stimulation portion. In the embodiment of FIG. 6, two control buttons 600, 605 are provided, with one button being usable to increase a value of, for example, a pulse width, current amplitude, and/or a frequency, of the electricity supplied to the stimulation portion, by a predetermined amount, and the other button being usable to decrease the value by a predetermined amount. As an example, the predetermined amount may be in respect to pulse with, and the increments may be set at increments of about 20 microseconds, in other words, the increasing or decreasing of the value using the control buttons 600, 605 may be an increase or decrease of 20 microseconds, from 0 to 200 microseconds (values may be 0, 20, 40, 60, 80, 100, 120, 140, 160, 180, and 200 microseconds). The buttons 600, 605 may include visual indicators of the function of the button, such as a plus symbol and a minus symbol, respectively. A user may increase the value up to a maximum value, and decrease the value down to a minimum value.

While the control buttons 600, 605 may provide for discrete adjustments by a predetermined amount, in other aspects, the control buttons 600, 605 may allow for control of probe 165 without the use of discrete positions or settings (i.e., continuous control). For example, buttons 600, 605 may allow a user to increase or decrease the value continuously along the range (e.g., from a value of 0 to 200).

One or more of the control buttons 145 discussed herein, including the ON/OFF button 180, the slider 500, the release button 515, and/or the control buttons 600, 605, may provide one or more of tactile feedback, auditory feedback, and/or display 150 may provide visual feedback to a user. The tactile feedback may be, for example, vibration, the auditory feedback may be, for example, a click, a tone, or another sound, and the visual feedback may be, for example, an indication provided on the display 150 of the handle 105. As more specific examples, the indication provided on the display 150 may include an “ON” display screen, an “OFF” display screen (which may include simply turning off the display 150), or a value of electrical supply, such as a selected value or an increase or decrease in value, that is input via one of the slider 500 or the control buttons 600, 605. As noted with respect to other embodiments, the value may be, for example, an electrode spacing, and/or a pulse width, a frequency, or an amplitude of the current, of the electricity supplied to the stimulation portion. As described previously, although ON/OFF button 180, the slider 500, the release button 515, and/or the control buttons 600, 605 are described in reference to particular actuator types for ease of reference, it is contemplated that any suitable type of actuator may be used in its place, e.g., one or more levers, triggers, knobs, sliders, buttons, control wheels, switches, etc. Further, any suitable number of actuators or displays may be incorporated on handle 105.

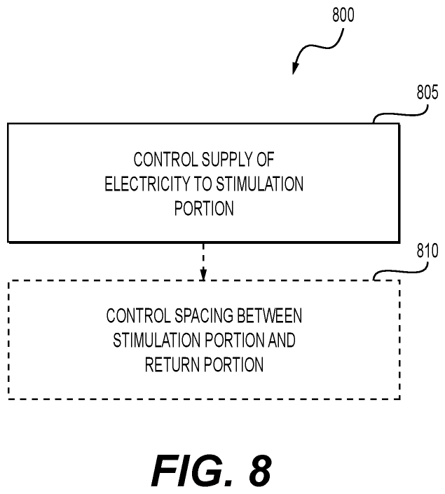

FIG. 7 is a schematic diagram of a controller 700, as the controller 155, for the electrical stimulation device 100 according to one or more embodiments. The controller 700 includes at least one memory or storage 705 that stores instructions, and at least one processor 710 configured to execute the instructions to perform a control method, described below with reference to FIG. 8. A timer 715 may also be provided as a part of the controller 700. The at least one memory 705, the at least one processor 710, and the timer may be provided on a printed circuit board (PCB), such that a single PCB arrangement may be used in manufacturing a controller 700 for use with various types of interchangeable probes for different purposes, such as nerve location, pain blocking, and nerve regenerating.

The controller 700 is configured to receive inputs 720 and to send outputs 725. For example, inputs 720 may include inputs from a user of the electrical stimulation device 100, including an input value, such as a pulse width, a current, a frequency, or an amplitude, of the electricity supplied to the stimulation portion. Inputs 720 may also include toggling of the ON/OFF control button 180, to turn the electrical stimulation device 100 on or off, a spacing input, to set, increase, or decrease the spacing S between the stimulation portion and the return portion, a duration input, setting a duration for supply of the electricity to the stimulation portion, and other inputs for the use of the electrical stimulation device 100. Outputs 725 may include output of electricity to the stimulation portion based on the input value, such as the pulse width, the current, the frequency, or the amplitude of the electricity. Outputs 725 may also include an instruction to the motor 160 to change the spacing S between the stimulation portion 170 and the return portion 175 of the probe 165 to the set value, the increased value, or the decreased value. The controller 700 may also send outputs 725 based on starting of the timer 715 and stopping of the timer 715 based on the input duration. One or more of the outputs 725 may be output to the display 150 for indication of the current settings or outputs to a user.

The controller 700 may be operably connected to the motor 160 within the handle 105, to control the spacing S between the stimulation portion 170 and the return portion 175 of the probe 165, for example. The controller 700 may also be connected to the first wire 120, to control the value of electrical parameters, such as a pulse width, a current amplitude, and/or a frequency, of electrical energy supplied to the stimulation portion 170 of the probe 165. And, as noted above, the controller 700 may provide outputs 725, including the outputs to the motor 160 and the first wire 120, based on an input duration of a pulse of supply of electricity to the stimulation portion 170 and operation of the timer 715. In other words, the controller 700 may control the duration of operation of the motor 160, the duration of therapy overall, and/or pulse duration using the timer 715. The range of time for the duration of the pulse of supply of electricity may be, for example, about 0 usec to about 200 usec. And the duration of operation of the motor 160 may be based on a change to the spacing S, for example, and the amount of time required to effect that change in the spacing S.

FIG. 8 is a flowchart of the control method 800, also referred to as a method of controlling the electrical stimulation device 100, according to one or more embodiments. The method 800 includes a step 805 of controlling supply of electricity to the stimulation portion 170 of the probe 165. In one or more embodiments, controlling the supply of electricity to the stimulation portion 170 of the probe 165 may include providing at least one of bipolar stimulation and monopolar stimulation, or both, to the stimulation portion 170 of the probe 165. In a case in which the electrical stimulation device 100 includes an ON/OFF button 180, controlling the supply of electricity to the stimulation portion 170 of the probe 165 may be based on switching of the ON/OFF button 180. That is, when the ON/OFF button 180 is toggled to ON, the controller 155 (700) may cause the electricity from the electrical supply 115 to be delivered to the stimulation portion 170 via the first wire 120. And, when the ON/OFF button 180 is toggled to OFF, the controller 155 (700) may cause the electricity from the electrical supply 115 not to be delivered to the stimulation portion 170 of the probe 165. In a case in which the control buttons 145 include the slider 500 or the control buttons 600, 605, controlling the electrical supply to the stimulation portion 170 of the probe 165 may include enabling changes via signal-control circuitry that is known in the art of electrical stimulation. As noted above with respect to other embodiments, the value may be, for example, a pulse width or an energy amount, such as an amplitude, of the electricity supplied to the stimulation portion.

As one example, in a case in which the electrical stimulation device 100 includes the stimulation portion 170 and the return portion 175 that extend parallel to one another, the method 800 may further include a step 810 of controlling the spacing S between the stimulation portion 170 and the return portion 175 of the probe 165 using the controller 155 (700). For example, the spacing S between the stimulation portion 170 and the return portion 175 of the probe 165 may be changed using the motor 160 that is connected to the stimulation portion 170 and/or the return portion 175. That is, the controller 155 (700) may operate the motor 160 to either increase or decrease the spacing S between the stimulation portion 170 and the return portion 175 of the probe 165. As another example, in a case in which the one or more control buttons 145 include a slider 500, controlling the spacing S between the stimulation portion 170 and the return portion 175 of the probe 165 includes moving the slider 500 in one of a distal direction and a proximal direction to cause the spacing S to change.

The electrical stimulation device 100 according to the embodiments discussed herein, and the related controller 155 (700) and control method (800) may be configured for use in one or more of locating one or more nerves, identifying one or more nerves, including identifying one or more of motor nerves and sensory nerves, assessing and/or diagnosing injuries to one or more nerves, stimulating regeneration of one or more nerves, providing a nerve block to one or more nerves, and managing and relieving nerve pain.

If desired, one or more aspects of the present disclosure may be used in combination with one or more other aspects. However, if desired, each aspect may be employed without other aspects of the disclosure.

Although electrical stimulation devices, controllers, and control methods of the present disclosure are described for use with nerves, examples of tissue with which electrical stimulation devices, controllers, and control methods described herein may be used include nerve tissue, such as peripheral nerve tissue or central nervous system tissue, as well as other types of tissue. Exemplary tissue types suitable for use with electrical stimulation devices and controllers of the present disclosure include, but are not limited to, epithelial tissue, connective tissue, muscular tissue, tendon tissue, ligament tissue, vascular tissue, intestinal tissue, dermal tissue, and cardiac tissue. The tissue may be mammalian tissue, including human tissue and tissue of other primates, rodent tissue, equine tissue, canine tissue, rabbit tissue, porcine tissue, or ovine tissue. In some aspects, the tissue may be non-mammalian tissue, selected from piscine, amphibian, or insect tissue. The tissue may be allogeneic or xenogeneic to a subject into which a graft is implanted. The tissue may be a synthetic tissue, such as, but not limited to, laboratory-grown or 3D-printed tissue.

It should be understood that although the present disclosure has been made with reference to preferred embodiments, exemplary embodiments, and optional features, modifications and variations of the concepts herein disclosed may be resorted to by those skilled in the art, and that such modifications and variations are considered to be within the scope of this disclosure as defined by the appended claims. The specific embodiments and examples provided herein are examples of useful embodiments of the present disclosure and are non-limiting and illustrative only. It will be apparent to one skilled in the art that the present disclosure may be carried out using a large number of variations of the devices, device components, methods, and steps set forth in the present description. As will be recognized by one of skill in the art, methods and devices useful for the present methods can include a large number of various optional compositions and processing elements and steps.

What is claimed is:

1. An electrical stimulation device comprising:
a handle including:
a proximal end;
a distal end;
one or more actuators; and
a display; and
a probe attached to and extending from the distal end of the handle, the probe including a stimulation portion and a return portion, wherein a spacing between the stimulation portion and the return portion is adjustable.

2. The electrical stimulation device of claim 1, wherein each of the stimulation portion and the return portion of the probe is a lead, and together, the leads form a single probe.

3. The electrical stimulation device of claim 1, wherein the probe is formed by the stimulation portion as a stimulation probe and the return portion as a return probe, and together, the probes form a double probe.

4. The electrical stimulation device of claim 1, wherein the stimulation portion and the return portion of the probe extend from the distal end of the handle parallel to one another.

5. The electrical stimulation device of claim 1, wherein the probe is interchangeable.

6. The electrical stimulation device of claim 5, wherein the handle includes a release button configured to detach the interchangeable probe.

7. The electrical stimulation device of claim 1, wherein the probe is a forceps probe attached to and extending from the distal end of the handle, the forceps probe functioning as a forceps.

8. The electrical stimulation device of claim 1, wherein the one or more actuators includes a slider, and movement of the slider causes the spacing between the stimulation portion and the return portion of the probe to change.

9. The electrical stimulation device of claim 1, wherein the one or more actuators includes a slider, and movement of the slider causes a change in one or more of:
a pulse width;
a current amplitude; or
a frequency,
of the electricity supplied to the stimulation portion.

10. The electrical stimulation device of claim 1, wherein the stimulation portion and the return portion of the probe are concentric.

11. The electrical stimulation device of claim 1, wherein the probe further includes an insulator portion provided between at least a portion of the stimulation portion and at least a portion of the return portion.

12. The electrical stimulation device of claim 1, wherein the device is capable of providing both:
monophasic stimulation in which the stimulation portion acts only stimulation probe and the return portion acts only as a return probe; and
biphasic stimulation in which the stimulation portion and return portion each act as stimulation and return probes in an alternating manner.

13. The electrical stimulation device of claim 1, wherein the device is configured to provide one or more of tactile feedback, auditory feedback, or visual feedback to a user.

14. The electrical stimulation device of claim 13, wherein the device is configured to provide tactile feedback as a vibration.

15. The electrical stimulation device of claim 1, wherein the one or more actuators include a button that, when depressed, causes the generation of one or more stimulation pulses.

16. The electrical stimulation device of claim 1, wherein the one or more actuators includes a set of control buttons configured to receive an input value to increase or decrease value by a predetermined amount, the input value being one of:
a pulse width;
a current amplitude; or
a frequency;
of the electricity supplied to the stimulation portion.

17. The electrical stimulation device of claim 1, wherein the display is a liquid crystal display (LCD).

18. The electrical stimulation device of claim 1, wherein the device is configured for use in one or more of the following:
locating one or more nerves;
identifying one or more nerves, including identifying one or more of motor nerves and sensory nerves;
stimulating regeneration of one or more nerves;
providing a nerve block to one or more nerves; and
managing and relieving nerve pain.
